(19) United States
(12) Patent Application Publication
(10) Pub. No.: US 2022/0223248 A1
(43) Pub. Date: Jul. 14, 2022

(54) A Medicine Compounding System

(71) Applicant: BOTANICAL RESOURCE HOLDINGS PROPRIETARY LIMITED, Somerset West (ZA)

(72) Inventors: Mark Kemp, Somerset West (ZA); Richard Paul Davies, Somerset West (ZA)

(21) Appl. No.: 17/613,989

(22) PCT Filed: May 22, 2020
(86) PCT No.: PCT/IB2020/054882

§ 371 Date: Nov. 24, 2021

(30) Foreign Application Priority Data
May 24, 2019 (ZA) 2019/03281

(51) Int. Cl. G16H 20/13; A61J 1/20; B01F 35/83; B01F 35/71; B01F 33/84; G16H 20/17

(52) U.S. Cl. CPC G16H 20/13; A61J 1/2089; A61J 1/2037; A61J 1/201; B01F 2101/22; B01F 35/7174; B01F 33/846; G16H 20/17; A61J 2205/10; B01F 35/83

(57) ABSTRACT

A medicine compounding system has a plurality of containers for containing medical compounds to be used to make up a medicine. A dosing chamber is connected to each of the plurality of containers and a drawing mechanism is used to draw compounds from the plurality of containers to the dosing chamber. A controller receives a script via a communications module which includes details of a patient and a medicine to be dispensed. The controller determines from the script which medical compounds and what quantities of each compound are to be used to form the medicine. The controller controls the drawing mechanism to draw the determined quantity of the medical compound from the container holding the compound into the dosing chamber, and once all of the required medical compounds have been drawn into the chamber to form the medicine, to eject the medicine formed in the dosing chamber via an outlet.

### BACKGROUND OF THE INVENTION

The present application relates to a medicine compounding system.

In particular, the present invention provides an automated process for the compounding of a medicinal product from a script produced by an authorised medical practitioner and dispensing thereof to a patient in need of the medicinal product.

Whilst compounding of medicines is an age old profession, the mass production of pharmaceutical products by large pharmaceutical manufacturers has meant that the compounding of specific medicines has been far less prevalent in recent decades.

However, patients have become more informed and have realised the limitations of mass produced pharmaceutical products, particularly in relation to active ingredient concentrations, dosage strengths, and the like, as well as in treating diseases and conditions not already addressed by the pharmaceutical industry. As a consequence, the requirement for healthcare practitioners to provide specific medicines tailored to treat specific ailments is once again on the increase. Coupled to this is a move away from synthetic pharmaceutical products to naturally derived products, which is the case with cannabinoid extracts, such as the cannabinoids delta-9-tetrahydrocannabinol (THC) and Cannabidiol (CBD), which have become increasingly more popular as their use is legalised around the world for medicinal purposes.

Traditionally compounding is carried out by compounding pharmacists under the direction of a qualified medical practitioner. In such a case, the medical practitioner draws up a script for the compounded medicine, which is then taken to a compounding pharmacist to formulate and dispense the medicine.

The present invention addresses this by providing an improved automated medicine compounding system.

### SUMMARY OF THE INVENTION

According to one example embodiment of the present invention there is provided a medicine compounding system comprising:
a communication module;
a plurality of containers for containing medical compounds to be used to make up a medicine to be dispensed to a patient;
a dosing chamber connected to each of the plurality of containers so that medical compounds can pass from the plurality of containers to the dosing chamber;
a drawing mechanism to draw compounds from the plurality of containers to the dosing chamber;
an outlet via which a medicine can be dispensed into a medicine container external to the system; and
a controller to receive a script via the communications module, the script including details of a patient and a medicine to be dispensed to the patient, wherein the controller determines from the script which medical compounds are to be used to form the medicine and what quantities of each medical compound is to be used, the controller further controlling the drawing mechanism to draw the determined quantity of the medical compound from the container holding the compound into the dosing chamber, and once all of the required medical compounds have been drawn into the chamber to form the medicine in the dosing chamber, to eject the medicine formed in the dosing chamber via the outlet.

The drawing mechanism may include a plunger located within the dosing chamber and movable within the dosing chamber and a stepper motor to move the plunger within the dosing chamber.

In one example, the plunger is also used to eject the medicine formed in the dosing chamber via the outlet.

The plunger may be moved in a first direction to draw medical compounds into the dosing chamber and is moved in a second direction to eject the medicine formed in the dosing chamber via the outlet.

The medicine compounding system preferably also includes a scanner, wherein the controller controls the scanner to scan the medicine either in the dosing chamber or in the medicine container and to compare the scanned medicine with the medicine received in the script to ensure the correct medicine is being dispensed to the patient.

The medical compounds may be in fluid form, wherein the dosing chamber is connected to each of the plurality of containers via one-way valves so that the medical compounds can flow from the plurality of containers to the dosing chamber but not from the dosing chamber to the plurality of containers.

The system may also include a dilution container in fluid communication with the dosing chamber via a one-way valve thereby allowing fluid diluent to flow from the dilution container to the dosing chamber but not from the dosing chamber to the dilution container.

In one example embodiment, the outlet is a dispensing needle, preferably an in-line mixing needle to ensure proper mixing of the compounds prior to dispensing.

### BRIEF DESCRIPTION OF THE DRAWINGS

FIG. 1 show a schematic diagram of a medicine compounding system according to the present invention;

### DESCRIPTION OF EMBODIMENTS

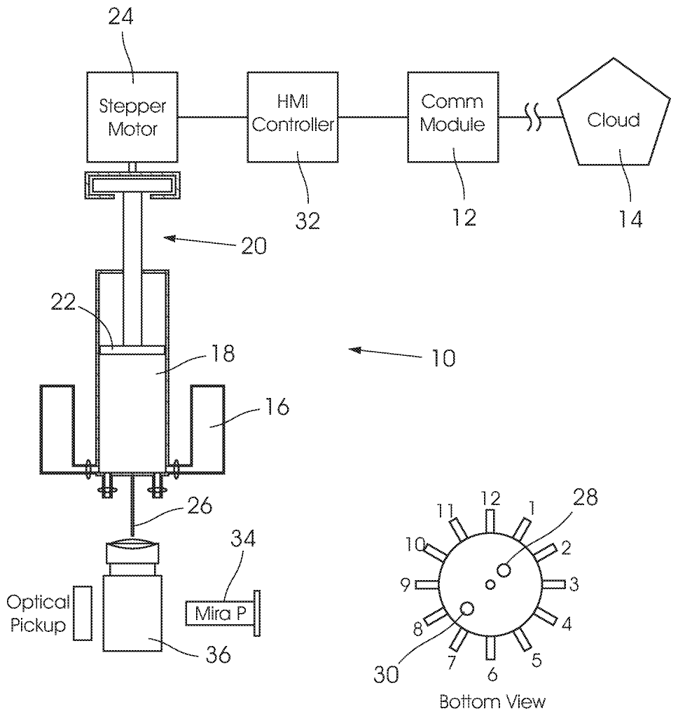

A medicine compounding system is provided, and an example of such a system 10 is shown in FIG. 1.

The system includes a communication module 12 which is used to communicate with a scripting system stored in a cloud 14.

Scripts for medicines to be produced will be downloaded from the cloud 14 as will be described in more detail below.

A plurality of containers 16 in use contain medical compounds to be used to make up a medicine to be dispensed to a patient.

Each container 16 will contain a defined content (such as a given cannabinoid, e.g. THC, or a given terpene). The content of each container 16 will be labeled and each container 16 will contain an RF (Radio Frequency) ID (Identification) Chip. The RF ID Chip associated with each container 16 will carry the data associated with the content of the given container 16 thus allowing the content of each container 16 to be “read” from the RF ID Chip. As each container 16 is connected to the manifold extending from dosing chamber 18, the RF ID Chip within each container 16 will be “read” by the medicine compounding system thus allowing the system to record the content and position of each container 16 connected to the manifold extending from dosing chamber 18. This configuration will allow the system to correctly select the containers 16 which contain the required medicine to be included in the final compound as prescribed by the prescribing medical practitioner.

A dosing chamber 18 is connected to each of the plurality of containers 16 so that medical compounds can pass from the plurality of containers 16 to the dosing chamber 18.

In one example embodiment, the dosing chamber 18 extends from a manifold which is surrounded by the containers 16.

In a prototype embodiment the medical compounds are in fluid form and the dosing chamber 18 is connected to each of the plurality of containers 16 via one-way valves so that the medical compounds can flow from the plurality of containers 16 to the dosing chamber 18 but not from the dosing chamber 18 to the plurality of containers 16.

A drawing mechanism 20 is used to draw compounds from the plurality of containers 16 to the dosing chamber 18.

In the example embodiment the drawing mechanism 20 includes a plunger 22 located within the dosing chamber 18 and movable within the dosing chamber, and a stepper motor 24 to move the plunger within the dosing chamber.

The system also includes an outlet 26 via which a medicine can be dispensed into a medicine container 36 external to the system.

The drawing mechanism 20 is used to both draw medical compounds into the dosing chamber 18 and then is also used to eject the medicine formed in the dosing chamber via the outlet 26.

Thus it will be appreciated that when the plunger is moved in a first direction to increase the volume inside the dosing chamber, medical compounds will be drawn into the dosing chamber 18 and when the plunger is moved in a second direction to decrease the volume inside the dosing chamber, the medicine formed in the dosing chamber will be ejected via the outlet 26. In one example embodiment the outlet 26 is a dispensing needle, particularly an in-line mixing needle to ensure proper mixing of the compounds prior to dispensing. Additional mixing means may be provided in some example embodiments.

The system also includes a dilution container and wash bottle (not shown) in fluid communication with the dosing chamber 18 via inlets 28 and 30 respectively and via one-way valves. These allow fluid diluent to flow from the dilution container and wash bottle to the dosing chamber 18 but not from the dosing chamber 18 to the dilution container and wash bottle.

3-way dump valves are also provided that allow for switching between the feed lines and a waste line (not shown).

A controller 32 typically includes a processor and software or firmware executing on the processor and controls the operation of the system.

The controller 32 controls the system to receive data including a script and to determine from the script which medical compounds are to be used to form the medicine, as well as what quantities of each medical compound is to be used.

The script could be imported into the system 10 in a number of different ways.

For example, the patient has with them a printed bar code or QR code representing the script which is presented to the system.

In this example the system includes a bar code or QR code reader.

The bar code or QR code is scanned and the data read is used to identify and request a script to be downloaded from the cloud 14.

In this example the script data is retrieved and loaded onto the dispensing system thereby reducing the need to manually capture the script data.

Alternatively, or in addition, if a participating dispensing pharmacy at which the system 10 is located is registered on a network and the patient has elected to have the pharmacy dispense the medicine, a link to the script in the script cloud repository 14 can be transmitted to the system.

The system 10 will then again be required to download the script in order to begin the process of dispensing.

It will be appreciated that in all cases the data including the script will be received at the system 10 in an encrypted format and the controller 32 will first need to decrypt the script data before processing can commence.

Once the script is obtained and decrypted, the controller 32 will validate the script to ensure compliance with relevant legislation, for instance FDA Regulation Title CFR 21 Part 11, which regulates Electronic Records and Electronic Signatures (ERES).

These regulations ensure that the ERES are trusted, reliable and equivalent to paper records.

Once validated, the controller 32 will control the drawing mechanism 20 to draw the determined quantity of the medical compound from the container 16 holding the compound into the dosing chamber 18.

When not in use, all of the containers 16 are dormant or locked, and only those that are linked to the script to be dispensed are activated, to ensure that only the ingredients selected by the medical practitioner in drawing up the script are capable of being dispensed.

For convenience, an example implementation of the present invention will be described with reference to the compounding of medicines containing medicinal cannabinoid extracts, but it will readily be understood by persons skilled in the art that the invention extends to any medicinal ingredients that are capable of being compounded and formulated into a medicine.

In this example, the selected cannabinoid extracts are drawn up in to the dosing chamber 18, in priority order (most potent to least potent).

The plunger 22, which is moved within the dosing chamber 18 by the stepper motor 24, is used to draw the medical compounds into the dosing chamber 18.

The stepper motor 24 is calibrated to ensure that a certain number of windings correspond to a given volume of medical compounds drawn up into the dosing chamber 18.

In order that only one medical compound is drawn in at a time, the one way valves are individually unlocked so that only the medical compound from one of the containers 16 is drawn into the dosing chamber 18 when the plunger is moved.

Once all of the required medical compounds have been drawn into the dosing chamber 18 it will be appreciated that the medicine required to be dispensed will be in the dosing chamber 18.

The different medical compounds may need to be mixed in the dosing chamber in which case a mixing mechanism is provided in the dosing chamber.

In the illustrated example, the in-line mixing needle ensures the proper mixing of the compounds prior to dispensing.

The controller 32 controls the stepper motor 24 to push the plunger 22 forward thereby to eject the medicine formed in the dosing chamber via the needle 26.

The system further including a scanner 34 which the controller 32 controls to scan the medicine either in the medicine container as illustrated or in the dosing chamber 18 prior to ejection.

The scanner 34, for example a Mira P device, is used to determine whether or not the medicine as formulated is indeed the medicine as compounded by the medical practitioner. In such a device, the scanning is carried out via Transmission Raman Spectroscopy that is capable of probing the entire contents of the sample in order to provide an accurate representation of the sample, be it in liquid, powder (including tablets and capsules) or other similar dosage forms.

The controller 32 then compares the scanned medicine with the medicine received in the script to ensure the correct medicine is being dispensed to the patient.

If validated, the medicine is digitally labelled and dispensed to the patient. If, however, the medicine fails the validation, it is rejected and not dispensed to the patient.

Whilst the diluent from the bottle or tank acts to flush out the system during use, the system in the shown embodiment includes a wash bottle or tank 30 for carrying out periodic deep cleaning of the system.

It will be appreciated that the example system 10 illustrated has been made as simple and cost effective as possible to allow broad access to the system by the dispensing pharmacy industry. However, it is to be understood that this is illustrative only, and more sophisticated systems that allow for simultaneous and multiple formulating and dosing operations can be employed.

It will be appreciated that the system and process provided ensure safe, secure, verifiable and auditable prescribing and dispensing of medicines.

Additionally, a number of systems 10 may be connected to a central server with each system 10 being registered to allow it to be monitored by the external server.

This will also allow for service and calibration and will allow for the cannabinoid elements to be monitored and restocked when defined minimum quantities are reached.

This allows stock planning, logistics planning, ordering protocols and stock replenishment at the point of dispensing, thus ensuring that the needs of the patient are always met.

1. A medicine compounding system comprising:
a communication module;
a plurality of containers for containing medical compounds to be used to make up a medicine to be dispensed to a patient;
a dosing chamber connected to each of the plurality of containers so that medical compounds can pass from the plurality of containers to the dosing chamber;
a drawing mechanism to draw medical compounds from the plurality of containers to the dosing chamber;
an outlet via which a medicine can be dispensed into a medicine container external to the system; and
a controller to receive a script via the communications module, the script including details of a patient and a medicine to be dispensed to the patient, wherein the controller determines from the script which medical compounds are to be used to form the medicine and what quantities of each medical compound is to be used, the controller further controlling the drawing mechanism to draw the determined quantity of the medical compound from the container holding the compound into the dosing chamber, and once all of the required medical compounds have been drawn into the chamber to form the medicine in the dosing chamber, to eject the medicine formed in the dosing chamber via the outlet.

2. A medicine compounding system according to claim 1 wherein the drawing mechanism includes a plunger located within the dosing chamber and movable within the dosing chamber and a stepper motor to move the plunger within the dosing chamber.

3. A medicine compounding system according to claim 2 wherein the plunger is used to eject the medicine formed in the dosing chamber via the outlet.

4. A medicine compounding system according to claim 3 wherein the plunger is moved in a first direction to draw medical compounds into the dosing chamber is moved in a second direction to eject the medicine formed in the dosing chamber via the outlet.

5. A medicine compounding system according to claim 1 further including a scanner, wherein the controller controls the scanner to scan the medicine either in the dosing chamber or in the medicine container and to compare the scanned medicine with the medicine received in the script to ensure the correct medicine is being dispensed to the patient.

6. A medicine compounding system according to claim 1 wherein the medical compounds are in fluid form.

7. A medicine compounding system according to claim 6 wherein the dosing chamber is connected to each of the plurality of containers via one-way valves so that the medical compounds can flow from the plurality of containers to the dosing chamber but not from the dosing chamber to the plurality of containers.

8. A medicine compounding system according to claim 6 further including a dilution container in fluid communication with the dosing chamber via a one-way valve thereby allowing fluid diluent to flow from the dilution container to the dosing chamber but not from the dosing chamber to the dilution container.

9. A medicine compounding system according to claim 1 wherein the outlet is a dispensing needle.

10. A medicine compounding system according to claim 9 wherein the dispensing needle is an in-line mixing needle to ensure proper mixing of the compounds prior to dispensing.
